(19) United States
(12) Patent Application Publication
(10) Pub. No.: US 2026/0151100 A1
(43) Pub. Date: Jun. 4, 2026

(54) COMPUTER-IMPLEMENTED METHOD FOR LOCATING AN OBJECT DETECTED IN A PLURALITY OF X-RAY PROJECTION IMAGES

(71) Applicant: Siemens Healthineers AG, Forchheim (DE)

(72) Inventors: Alois Regensburger, Poxdorf (DE); Bernd Niedermeyer, Effeltrich (DE)

(21) Appl. No.: 19/404,554

(22) Filed: Dec. 1, 2025

(30) Foreign Application Priority Data
Dec. 3, 2024 (DE) 10 2024 211 540.1

(51) Int. Cl. A61B 6/00; A61B 6/03; A61B 6/46; G06V 10/25; G06V 10/774; G06V 10/82; G06V 20/50; G06V 20/64; G06V 20/70; G16H 30/40

(52) U.S. Cl. CPC A61B 6/5217; A61B 6/466; A61B 6/5235; G06V 10/25; G06V 10/774; G06V 10/82; G06V 20/50; G06V 20/64; G06V 20/70; G16H 30/40; A61B 6/032; G06V 2201/033

(57) ABSTRACT

A computer-implemented method for providing an item of detection information includes applying a function to a received first X-ray projection image, at least one received second X-ray projection image, and a received and/or ascertained item of relative arrangement information. The function is configured to identify one projection respectively from at least one object in a predefined epipolar region in the first X-ray projection image and in the at least one second X-ray projection image. The predefined epipolar region is arranged around an epipolar line on which the projection of the object is situated in the first X-ray projection image and/or in the at least one second X-ray projection image. A corresponding item of detection information is provided when the projection of the object is identified in the predefined epipolar region in the first X-ray projection image and in the at least one second X-ray projection image.

The present patent document claims the benefit of German Patent Application No. 10 2024 211 540.1, filed Dec. 3, 2024, which is hereby incorporated by reference in its entirety.

### TECHNICAL FIELD

The disclosure relates to a computer-implemented method for providing an item of detection information. The item of detection information describes at least one object detected in a plurality of X-ray projection images. The disclosure also relates to a computer-implemented method for providing a learned function, a method for providing an item of detection information, a data processing system for carrying out the computer-implemented method, and a computer program product for carrying out the computer-implemented method.

### BACKGROUND

Individual two-dimensional X-ray projection images, for example, which describe at least one part of a patient from different perspectives, may be captured with an X-ray imaging system. For example, two or more individual X-ray projection images may be captured, e.g., with a relative arrangement between the X-ray imaging system and the patient being changed between the capturing of these X-ray projection images. Such individual X-ray projection images of the patient are frequently captured by two-dimensional fluoroscopy. However, compared with sectional images captured, for example, by computed tomography (CT), this kind of capture of X-ray projection images is less suitable for identifying small structures, such as a bone fragment, which has chipped off a bone after a fracture, or internal bleeding. The reason for this is that such small structures may only be identified with difficulty in the two-dimensional X-ray projection images. This may result in incorrect or unsuitable decisions on therapy, which may subsequently still result in a laborious check via CT.

However, compared with capturing the individual X-ray projection images, a CT scan is time-consuming and linked to an additional X-ray dose for the patient. It is therefore expedient to automatically ascertain whether, on the basis of a plurality of individual X-ray projection images, indications exist that a, for example, three-dimensional control examination may be expedient, for example, by volume tomography (Cone Beam Computed Tomography, CBCT), a C-arm X-ray imaging system for capturing three-dimensional X-ray projection images, or CT. Therefore, the small structure or object may be sought in a plurality of X-ray projection images.

### SUMMARY AND DESCRIPTION

It is the object of the disclosure to provide a solution by which a plurality of X-ray projection images may be examined with regard to the presence of an object.

The scope of the present disclosure is defined solely by the appended claims and is not affected to any degree by the statements within this summary. The present embodiments may obviate one or more of the drawbacks or limitations in the related art.

A first aspect of the disclosure relates to a computer-implemented method for providing an item of detection information. The item of detection information describes at least whether at least one object is identified in a plurality of X-ray projection images. It may describe that the object image is or is not identified in the plurality of X-ray projection. In addition, the item of detection information may describe the at least one object identified and thus detected in a plurality of X-ray projection images. The item of detection information may therefore describe, for example, that a specific object detected in a first X-ray projection image is also detected in at least one second X-ray projection image. In addition to the presence of the detected object, the item of detection information may describe a position and/or orientation of the detected object. In one example, the item of detection information may describe that no object is identified in the plurality of X-ray projection images. In this example, the at least one object was not identified in the first X-ray projection image and/or in the at least one second X-ray projection image. Providing the item of detection information may include that the presence or absence of the object is signaled. Further, the detected object may be displayed on the basis of the provided item of detection information, for example, in that it is marked and/or annotated and/or highlighted in the X-ray projection images. In an example, the object is a small structure in a body of a patient, such as a bone fragment, internal bleeding, a foreign body, in particular an implant, a contrast agent, for example, in a vessel, and/or a different kind of object, in which an individual, for example, a physician, is interested and/or is predefined as the object of interest.

The computer-implemented method includes receiving a first X-ray projection image of the plurality of X-ray projection images and at least one second X-ray projection image of the plurality of X-ray projection images. For example, numerous second X-ray projection images may be received. The received X-ray projection images map an at least partially common examination region of an examination object from different directions of projection. The received X-ray projection images were therefore captured from mutually different perspectives. The X-ray projection images are, in particular, two-dimensional projection images respectively and thus two-dimensional X-ray projection images. The first X-ray projection image and the at least one second X-ray projection image describe or show, for example, a body part of a patient from mutually different perspectives, i.e., from mutually different directions of projection. In this example, the body part is the examination region and the patient is the examination object. For example, an X-ray imaging system that captures the X-ray projection images may have been moved relative to the patient and/or the patient may have been moved relative to a stationary X-ray imaging system for this purpose, while the plurality of X-ray projection images were captured, which are received in the framework of the computer-implemented method. Alternatively, the X-ray projection images may be referred to as X-ray images. The respective received X-ray projection image may be a pre-prepared X-ray projection image.

The computer-implemented method includes receiving and/or ascertaining an item of relative arrangement information. The item of relative arrangement information describes a relative arrangement of the different directions of projection. It thus describes a relationship between the direction of projection from which the first X-ray projection image was captured, and the direction of projection from which the at least one second X-ray projection image was captured. The item of relative arrangement information may be understood as an item of information or as data, which describes a relative projection geometry of the different directions of projection. The item of relative arrangement information may be received, for example, if the X-ray imaging system captures a movement of an X-ray projection image recording facility of the X-ray imaging system relative to the patient. For example, in addition to the individual X-ray projection images, a C-arm X-ray imaging system may indicate in which positioning of the C-arm the respective X-ray projection image was captured. Items of information of this kind on the positioning of the C-arm, which may alternatively be referred to as angulation data, are received in one example in the framework of the computer-implemented method as the item of relative arrangement information.

If the item of relative arrangement information is ascertained, it is possible, for example, using machine learning methods, that is to say a trained machine learning model, using the received plurality of X-ray projection images, to ascertain a three-dimensional body part model, in particular a three-dimensional bone model. The relative projection geometry may then be derived from the three-dimensional body part model and thus the item of relative arrangement information may be ascertained. Alternatively, or in addition, it is possible to ascertain a three-dimensional model of the patient during capture of the respective X-ray projection image by at least one camera, which at least partially captures the patient during capture of the X-ray projection images. The item of relative arrangement information may be at least estimated, in particular ascertained, on the basis of the three-dimensional model. Known methods for ascertaining the item of relative arrangement information may be drawn on here.

It may be assumed that when carrying out the computer-implemented method, at least two different X-ray projection images and items of information relating thereto exist, from which perspectives these X-ray projection images were captured relative to one another, so, for example, calculations are possible in order to be able to find a predefined location in the first X-ray projection image, at least estimated, in particular exactly, in the at least one second X-ray projection image.

The computer-implemented method includes applying a function to the received first X-ray projection image, the at least one received second X-ray projection image and the received and/or ascertained item of relative arrangement information as input data. The function may be embodied as a learned or trained function. Alternatively, the function may be an unlearned or untrained function that may be understood as a rule-based algorithm. The learned function may be understood as a learned or trained machine learning model. The learned function may be embodied, for example, as a trained neural network module, which includes at least one artificial neural network. In an example, the trained neural network module includes at least one convolutional neural network (CNN).

The function is embodied to identify one projection respectively of at least one object in a predefined epipolar region in the first X-ray projection image and in the at least one second X-ray projection image. When the function is applied, one projection respectively of the at least one object may be sought, that is to say identified, in a predefined epipolar region, for example, in the first X-ray projection image as well as in the at least one second X-ray projection image. The predefined epipolar region is arranged around an epipolar line on which the projection of the at least one object is situated in the first X-ray projection image and/or in the at least one second X-ray projection image. For example, the at least one object is sought in all X-ray projection images, to which the function is applied, in parallel, in particular simultaneously, in order to identify the at least one object in these X-ray projection images or in at least one individual X-ray projection image of these X-ray projection images. If, the projection of the at least one object is identified or detected, that is to say found, for example, in the first X-ray projection image, the epipolar line on which the detected projection is situated is ascertained, and the associated epipolar region around the epipolar line is ascertained in the at least one second X-ray projection image and it is checked, for example, whether the projection of the at least one object, which was identified or detected in the at least one second X-ray projection image, is situated in this epipolar region. If the projection of the at least one object is detected, that is to say identified or found, in another example or also in the at least one second X-ray projection image, the epipolar line on which the detected projection is situated is ascertained, and the associated epipolar region around the epipolar line is ascertained in the first X-ray projection image and it is checked, for example, whether the projection of the at least one object, which was detected in the first X-ray projection image, is situated in this epipolar region. The projection of the at least one object in the respective X-ray projection image may be understood as a shadow of the object in the respective X-ray projection image.

The computer-implemented method includes providing the item of detection information as output data of the function. The provided item of detection information describes that the at least one object is identified in a plurality of X-ray projection images if the projection of the at least one object in the predefined epipolar region was identified in the first X-ray projection image and in the at least one second X-ray projection image. If the projection of the at least one object is found in the predefined epipolar image region in the first X-ray projection image and in the at least one second X-ray projection image, for example, the item of detection information is provided that describes at least the detection of the at least one object and possibly also the detected object. It may then, for example, be output that the object was detected in a plurality of X-ray projection images. The item of detection information is suitable, for example, for checking whether a chipped bone fragment is found in a plurality of X-ray projection images, and thus it may be inferred that this bone fragment actually exists and it is not, for example, a misinterpretation of the first X-ray projection image. An automatic capture of an object takes place in a plurality of X-ray projection images, with it being possible to discover even smaller structures, such as the chipped bone fragments. However, it is not already assumed after a one-time detection that the object exists, rather firstly it is automatically ascertained, in particular checked, whether this discovery, which is to say the object, may be found in a plurality of X-ray projection images and thus the discovery may be confirmed. The item of detection information is provided only if an automatically detected object occurs in a plurality of X-ray projection images. Individual X-ray projection images may thus be examined with regard to the presence of an object.

One exemplary embodiment includes that the function has a first object-ascertaining algorithm and a second object-ascertaining algorithm or was taught in a training process on the basis of the first object-ascertaining algorithm and the second object-ascertaining algorithm. Applying the function includes ascertaining a first item of object information. The first item of object information is ascertained by applying the first object-ascertaining algorithm to the first X-ray projection image. The first item of object information describes at least one object that is detected in the first X-ray projection image. The first object-ascertaining algorithm may include guidelines on what kind of object is being sought in the first X-ray projection image. Alternatively, or in addition, any object may be sought in the first X-ray projection image. In an example, a chipped bone fragment is detected as the at least one object in the first X-ray projection image. Detecting the at least one object means that a projection and thus a shadow of the object is detected in the X-ray projection image. The first object-ascertaining algorithm includes at least one rule and/or provision, the implementation of which, on the basis of at least the first X-ray projection image, makes it possible for the at least one object to be detected and thus identified or located. When the first object-ascertaining algorithm is applied, the object may be detected and thus identified, for example, on the basis of methods of pattern recognition and/or by a segmentation, in particular a threshold-based segmentation.

Applying the function includes ascertaining a second item of object information by applying a second object-ascertaining algorithm to the at least one second X-ray projection image. In an example, the second object-ascertaining algorithm is different from the first object-ascertaining algorithm, i.e., two different object-ascertaining algorithms exist. The second item of object information may be only ascertained when the first item of object information was previously ascertained, i.e., when the at least one object may be detected in the first X-ray projection image. When the second object-ascertaining algorithm is applied, the object already identified in the first X-ray projection image may be detected, and may thus be identified in the at least one second X-ray projection image, for example, on the basis of methods of pattern recognition and/or by a segmentation, in particular a threshold-based segmentation.

The second object-ascertaining algorithm is applied not only to the at least one second X-ray projection image, but also to the ascertained first item of object information and the received and/or ascertained item of relative arrangement information. Not only is the second X-ray projection image, in which the object is to be sought, therefore available to the second object-ascertaining algorithm, but items of information about the object that has already been detected as well as the relative arrangement between the received X-ray projection images are also available to it.

The second item of object information describes that the at least one object, which was detected in the first X-ray projection image, is also detected in the at least one second X-ray projection image. The object detected in the first X-ray projection image is therefore purposefully sought in the at least one second X-ray projection image with the second object-ascertaining algorithm. The second object-ascertaining algorithm is configured to ascertain at least one three-dimensional model of the at least one object on the basis of a projection of the at least one object in the first X-ray projection image. This three-dimensional model may alternatively be referred to as a three-dimensional object model. In an example, a plurality of three-dimensional models is ascertained, which take into account, for example, different three-dimensional embodiments of the object, which may be ascertained starting from the projection of the object in the first X-ray projection image. It is therefore estimated or predicted how the object detected in the first X-ray projection image may look three-dimensionally.

The second object-ascertaining algorithm is embodied to identify a projection of the at least one ascertained three-dimensional model in a predefined epipolar region in the at least one second X-ray projection image. For this purpose, the projection of the at least one ascertained three-dimensional model may be sought, for example, in a predefined epipolar region in the at least one second X-ray projection image. In an example, projections of a plurality of, in particular all, ascertained three-dimensional models are sought in the predefined epipolar region. The predefined epipolar region is a portion of the at least one second X-ray projection image in which the projection of the at least one ascertained three-dimensional model is expected. It is assumed here that the three-dimensional form of the object, that is to say the three-dimensional model, is at least estimated, in particular ascertained, on the basis of the projection of the object in the first X-ray projection image, so it is possible to at least estimate, in particular predict where and in what form the object is expected in the second X-ray projection image, (e.g., where the object may be arranged), if the object in the second X-ray projection image is the object from the first X-ray projection image.

The second object-ascertaining algorithm includes, for example, at least one rule and/or provision, on the implementation of which, on the basis of the at least one second X-ray projection image, the first item of object information and the item of relative arrangement information is ascertained as to whether an object matching or correlating with the object detected in the first X-ray projection image may also be ascertained in the at least one second X-ray projection image, so then the object is detected in the first X-ray projection image as well as in the at least one second X-ray projection image. It is therefore not any object that is sought in the second X-ray projection image, rather precisely the object that is detected in the first X-ray projection image. Corresponding or matching objects are thus sought.

If the projection of the at least one ascertained three-dimensional model is found in the at least one second X-ray projection image, the at least one object is also detected in the at least one second X-ray projection image. In this case, the second item of object information is ascertained that describes that the at least one object, which was detected in the first X-ray projection image, is or was also detected in the at least one second X-ray projection image. However, if it is established, for example, that no projection of the at least one ascertained three-dimensional model is found in the predefined epipolar region in the at least one second X-ray projection image, the at least one object is not detected in the at least one second X-ray projection image.

If a plurality of three-dimensional models is ascertained, projections of the plurality of three-dimensional models are sought in the predefined epipolar region. In one example, it is then sufficient if a single projection relating to a single one of the plurality of ascertained three-dimensional models is found in the at least one second X-ray projection image in order to assume from this that the object is also detected.

Applying the function includes that the item of detection information, which describes that the at least one object is identified in a plurality of X-ray projection images, is provided only if the first item of object information as well as the second item of object information are ascertained. As the projection of the at least one object is sought in the first X-ray projection image and a discovery of the at least one object is checked in that the object is sought in the epipolar region in the at least one second X-ray projection image, the object may be sought and detected quickly and reliably in a plurality of X-ray projection images. This may reliably prevent, for example, projections of different objects in the X-ray projection images from being wrongly interpreted as a common object.

Another exemplary embodiment provides that an epipolar line is ascertained on which the projection of the at least one object is situated in the first X-ray projection image. An epipolar line, on which the projection of the at least one object is situated, is therefore ascertained for the at least one second X-ray projection image for a detected object in the first X-ray projection image. When the second object-ascertaining algorithm is applied, the predefined epipolar region in the at least one second X-ray projection image is arranged around the ascertained epipolar line. The epipolar line indicates in which portion of the at least one second X-ray projection image the object is to be sought, which was detected in the first X-ray projection image. The epipolar line may be understood as a line along that a projection of an object must be arranged in a plurality of projection images if the projection images describe the object from different perspectives. Epipolar geometry is drawn on for this purpose, which is to say a mathematical model that describes geometric relationships between different mappings of the same object from different relative perspectives. The epipolar geometry allows a dependence between corresponding image points to be found in different X-ray projection images, with the image points corresponding when they describe a common object point of the object to be mapped in the different X-ray projection images. In other words, it is computationally ascertained where approximately the object may be arranged in the second X-ray projection image for it to be the same object, which was detected in the first X-ray projection image, with the epipolar line being ascertained and taken into account for this purpose. The specification of the epipolar line restricts a search region in the at least one second X-ray projection image, although to exactly the region in which the projection of the object must be situated. Objects, which are detected in the second X-ray projection image outside of the predefined epipolar region around the epipolar line, cannot be attributed to the object, which was detected in the first X-ray projection image. In other words, they do not match this object but are different from the object. For example, an expenditure of time of the second object-ascertaining algorithm may be significantly reduced compared to a procedure in which the entire at least one second X-ray projection image is searched through for the object. Furthermore, the number of false and/or false-positive detection events may be reduced in the second X-ray image compared to a procedure in which the entire at least one second X-ray projection image is searched through for the object.

A further exemplary embodiment includes that the predefined epipolar region extends, at least on one side of the epipolar line, up to a boundary line. The epipolar line and the boundary line run, for example, parallel to one another. In an example, the predefined epipolar region extends around the epipolar line on both sides up to a boundary line, respectively. Two boundary lines may then be provided that run parallel to the epipolar line but spatially offset to it, respectively. A distance between the epipolar line and the boundary line is strictly predefined. In certain examples, a predefined number of pixels or image points may be situated between the epipolar line and the boundary line and thus predefine a width of the predefined epipolar region at least on one side of the epipolar line. The strictly predefined distance may be dependent on an accuracy with which a relative arrangement of the patient to the X-ray imaging system may be ascertained during capture of the X-ray projection images. Alternatively, or in addition, inaccuracies in ascertaining the epipolar line and/or locating the object in the first X-ray projection image may be taken into account to be able to expediently select the distance between the epipolar line and the boundary line. The idea here is that this distance is large enough for the entire epipolar region to be encompassed in the X-ray projection image in which the object may be arranged if it may be detected in the at least one second X-ray projection image.

As an alternative to the strictly predefined distance, the distance between the epipolar line and the boundary line may be dependent on a size of the projection of the at least one ascertained three-dimensional model. The size of the projection may be a diameter, a height, a width, and/or a depth of the three-dimensional model. For example, the distance on one side between the epipolar line and the boundary line may correspond to at least half of the size of the projection, for example, in the case of boundary lines arranged on both sides of the epipolar line, or the size of the projection, for example, in the case of the boundary line arranged on one side of the epipolar line. In addition, a tolerance width may be taken into account in addition to the size of the projection in order to predefine the distance. If a plurality of three-dimensional models are ascertained, the distance may be predefined while taking into account the maximum size of the projection of the different three-dimensional models. The predefined epipolar region around the epipolar line may therefore be dynamically adjusted to the object. Therefore, an object-dependent dimensioning of the predefined epipolar region may take place. This enables reliable detection of the object in the at least one second X-ray projection image since a suitable section of the second X-ray projection image is searched through.

According to an additional exemplary embodiment, after the first X-ray projection image has been received, at least one detection image region is ascertained by applying a detection image region-ascertaining algorithm to the first X-ray projection image. The detection image region may therefore be ascertained even before the first object-ascertaining algorithm is applied. The ascertained at least one detection image region predefines at least one image region of the first X-ray projection image for applying the first object-ascertaining algorithm. The ascertained at least one detection image region describes, for example, at least one image region of the first X-ray projection image, in which the at least one object is to be searched for, (i.e., sought), when the first object-ascertaining algorithm is applied. The first object-ascertaining algorithm is applied to the ascertained at least one detection image region. In an example, the first object-ascertaining algorithm is only applied to the ascertained at least one detection image region and, for example, not to other image regions in the first X-ray projection image. An image region may be understood as a region as was described above in connection with the second X-ray projection image, i.e., as a portion or section of the first X-ray projection image.

In other words, a pre-selection of at least one area of interest or region of interest in the first X-ray projection image takes place on the basis of the detection image region in which image the object is subsequently purposefully sought. The detection image region may alternatively be understood as an anatomical search region for a detection task, i.e., for applying the first object-ascertaining algorithm. The detection image region may be an environment of a bone in which the bone adjoins tissue, a region of a joint, and/or an environment of a fracture that may be automatically identified in the first X-ray projection image.

The detection image region may be automatically ascertained in that, for example, at least one area or region of interest is strictly predefined that is subsequently ascertained in the first X-ray projection image. In other words, the image regions in the first X-ray projection image are ascertained, which may be assigned or correspond to the at least one area or region of interest. Alternatively, or in addition, the detection image region may be manually predefined by the individual, e.g., by the physician or another medical member of staff. For example, a manual input by an input apparatus may be captured for this purpose, and this may be assigned to the X-ray imaging system and/or a data processing system, which carries out the computer-implemented method. In an example, the detection image region is ascertained automatically.

The detection image region-ascertaining algorithm includes, for example, at least one rule and/or provision, on the implementation of which it is ascertained, on the basis of the first X-ray projection image, where the at least one detection image region is arranged in this image. Items of information on detection tasks and thus a list of detection image regions, for example, of areas of interest or regions of interest, may be available to the detection image region-ascertaining algorithm for this purpose. It is possible to automatically establish where in the first X-ray projection image the object may actually be searched for. This reduces computing effort as well as the time expenditure for carrying out of the computer-implemented method and results, moreover, in a pre-selection that may prevent non-relevant objects, such as intact bone segments, from being ascertained.

Another exemplary embodiment includes that after the first X-ray projection image has been received, a three-dimensional body part model is ascertained by applying a body part model-ascertaining algorithm to the first X-ray projection image. The ascertained three-dimensional body part model three-dimensionally describes a part of a body of the examination object mapped by the first X-ray projection image. If, for example, a leg of a patient is shown on the first X-ray projection image, the ascertained three-dimensional body part model describes this leg of the patient in three dimensions.

The body part model-ascertaining algorithm includes, for example, at least one rule and/or provision, on the implementation of which the three-dimensional body part model is generated and thus ascertained. In other words, a three-dimensional model of a bone anatomy of a patient may therefore be generated on the basis of the first X-ray projection image. In one example, the body part model-ascertaining algorithm is applied not only to the first X-ray projection image, but also to the at least one second X-ray projection image in order to ascertain the three-dimensional body part model.

The ascertained three-dimensional body part model is taken into account when the detection image region-ascertaining algorithm is applied. It may therefore be assumed that when ascertaining the detection image regions, it is known which body part is described by the first X-ray projection image and how this is expected to look in three dimensions. For example, soft tissue around bone, regions of a joint, or regions around visible fractures, which may be marked in the three-dimensional body part model, may automatically be identified particularly quickly and easily hereby. The detection image region may be quickly ascertained hereby, since details on body part described in the first X-ray projection image are taken into account.

A further exemplary embodiment includes that after the second item of object information has been ascertained, an item of probability information is ascertained. The item of probability information describes a probability with which the respective projection of the object is in the first X-ray projection image and in the at least one second X-ray projection image an object from a predefined group of objects of interest. It is therefore checked whether the object detected in the first X-ray projection image is an object of interest, i.e., a candidate for an object that is relevant, for example, when the item of detection information is provided. The item of probability information may be ascertained here, for example, by applying an algorithm for ascertaining the item of probability information, with it being possible for the algorithm for ascertaining the item of probability information to include at least one rule and/or provision, on the implementation of which the probability is ascertained. The item of probability information may be understood as a kind of plausibility that indicates whether an object is actually a plausible candidate for providing the item of detection information, or not. For example, the group of objects of interest may include bone fractures, bone splinters, implants, medical instruments, catheters, tips of catheters, other foreign bodies, contrast agent, and/or internal bleeding. The group of objects of interest does not include, for example, undamaged bone. In this example, the item of probability information may therefore describe, for example, how probable it is that the detected object in the first X-ray projection image shows a chipped bone fragment and not an intact bone.

Only if the ascertained item of probability information is situated in a predefined probability value range does the function provide the item of detection information that describes that the at least one object is identified in a plurality of X-ray projection images. As an alternative, the function predefines the provided item of detection information depending on the ascertained item of probability information. Alternatively, or in addition, the provided item of detection information includes the ascertained item of probability information. For example, an item of probability information of zero may be predefined for a minimum probability that an object is assigned to the group of objects of interest. An item of probability information of 1 or 100 may then describe a maximum probability that the object is assigned to the group of objects of interest. In this example, the probability value range may define at least one lower threshold value and an upper threshold value, above which or up to which the item of probability information is large enough to provide the item of detection information. Other definitions of the item of probability information and of the probability value range are possible.

By ascertaining and taking into account the item of probability information it is possible to prevent objects being sought in the X-ray projection images, which are not actually of interest, such as intact portions of bone. This makes it possible to focus on strictly or dynamically predefined types of objects that are assigned to the group of objects of interest. This shows how a purposeful search for chipped bone fragments is possible, without objects being detected in the process that, owing to their position, are most probably not chipped bone fragments.

In another exemplary embodiment, a three-dimensional body part model is ascertained by applying a body part model-ascertaining algorithm to at least the first X-ray projection image. The three-dimensional body part model may be ascertained as described above here. The ascertained three-dimensional body part model therefore three-dimensionally describes a part of a body of the examination object mapped by the first X-ray projection image. The at least one object is registered with the ascertained three-dimensional body part model. For example, the at least one object described by the first item of object information is therefore marked in the ascertained three-dimensional body part model. The three-dimensional body part model ascertained here therefore includes items of information on the detected object, for example, its position and/or orientation relative to other parts of the body part model.

In addition, at least one arrangement region respectively in the body is predefined for the objects in the predefined group of objects of interest. For example, this is entered in a list for the individual objects of interest in the group of objects of interest. For example, this may include that areas around bones, but not inside bones, are predefined for chipped bone fragments, whereas the contrast agents may be expected in vessels. Other and/or further arrangement regions than these examples are possible.

When ascertaining the item of probability information, it is taken into account whether an arrangement of the object registered in the ascertained three-dimensional body part model is at least partially situated in at least one of the arrangement regions that is predefined for at least one of the objects in the predefined group of objects of interest, or not. On the basis of the position and/or orientation of the detected object in the three-dimensional body part model and thus in the body of the patient, it is therefore estimated how probable it is that an object of interest is situated in this arrangement relative to the body part model, with the predefined arrangement regions being drawn on for this purpose. It may hereby reliably be estimated whether a detected object may be examined further in that it is sought, for example, in the second X-ray projection image, or whether the object may no longer be taken into account hereinafter. This results in a discarding of objects that are not sufficiently relevant, without the entire method, in particular providing the item of detection information, actually having to take place for each of these objects. This results in an appropriate selection of potential objects.

The described procedure may be based on the finding that a chipped bone fragment cannot be situated inside a bone, but only outside of the bone in the vicinity of the bone, since there is soft tissue around the bone, which may spatially restrict wandering of the chipped bone fragment. Locations inside the body of the patient are thus known at which a predefined object of interest, such as the chipped bone fragment, may be arranged, but also locations at which it may not be arranged. The items of information on the arrangement of predefined types of objects in the body are accordingly used in order to keep, for example, computing effort and time expenditure of the computer-implemented method low since objects may be rejected early as not being relevant or not being of interest.

Furthermore, one exemplary embodiment provides that after ascertaining the first item of object information, a third object-ascertaining algorithm is applied to the first X-ray projection image. An epipolar line is ascertained on which the projection of the at least one object is situated in the first X-ray projection image. It is then ascertained whether at least one projection of a further object, which corresponds with the at least one object, is arranged in the predefined epipolar region around the epipolar line. The above-described ascertained epipolar line may be ascertained here when the third object-ascertaining algorithm is applied in order to ascertain the epipolar line on which the detected object is situated in the first X-ray projection image. The epipolar line described here may therefore correspond to the above-described epipolar line. If the at least one projection of the further object is ascertained, which is situated along this epipolar line, no item of detection information is provided. Alternatively, the item of detection information may be provided that describes that the at least one object is not identified in a plurality of X-ray projection images.

Alternatively, if the at least one projection of the further object was ascertained, it may be checked whether the projection of the at least one object or the projection of the further object may be assigned to the found projection of the at least one ascertained three-dimensional model in the at least one second X-ray projection image. If the projection of the at least one object or the projection of the further object may be assigned to the found projection of the at least one ascertained three-dimensional model in the at least one second X-ray projection image, the item of detection information is provided. In order to assign the projection of the at least one object or the projection of the further object to the found projection of the at least one ascertained three-dimensional model in the at least one second X-ray projection image, an item of probability information is ascertained that describes with which probability the projection of the at least one object or the projection of the further object on the one hand and the found projection of the at least one ascertained three-dimensional model in the at least one second X-ray projection image describe the same object. If the item of probability information ascertained in this connection is situated in a predefined probability information region, the projection of the at least one object or the projection of the further object is deemed assigned to the found projection of the at least one ascertained three-dimensional model in the at least one second X-ray projection image. However, if the ascertained item of probability information is not situated in the predefined probability information region, the projection of the at least one object or the projection of the further object is deemed to not be assigned to the found projection of the at least one ascertained three-dimensional model in the at least one second X-ray projection image.

In other words, a search takes place along the epipolar line in the first X-ray projection image as to whether, in addition to the detected object, there are further objects that are situated along the epipolar line. If this is the case, i.e., further objects are detected along the epipolar line, it may not be ruled out that an object detected in the predefined epipolar region in the at least one second X-ray projection image may really be assigned to the originally detected object and not to one of the other corresponding objects along the epipolar line. In the case of a plurality of objects along the epipolar line, the object in the second X-ray projection image may no longer be unambiguously assigned to an object in the first X-ray projection image. However, this is only the case if the object and the further object correspond with one another, i.e., the connection satisfies a predefined minimum similarity. This may include that two objects may be assigned to a common type of object. This is the case, for example, if the object and the further object are both bone splinters. If, therefore, a plurality of objects of the same type are ascertained along the epipolar line in the first X-ray projection image, the object along the epipolar line in the second X-ray projection image may no longer be unambiguously assigned to the object. The second item of object information would then not be meaningful, for example. It is therefore possible to dispense with ascertaining the item of detection information. The computer-implemented method may be stopped directly or carried out for a different object in the first X-ray projection image, which is not situated on the epipolar line. Alternatively, the item of detection information is provided that describes that the at least one object is not identified in a plurality of X-ray projection images. This is an additional safeguarding mechanism for preventing the provision of inappropriate, since potentially incorrect, items of detection information.

A further exemplary embodiment provides that, if the projection of the at least one ascertained three-dimensional model is not found in the at least one second X-ray projection image, no second item of object information is ascertained and thus no item of detection information is provided. It is only on the basis of an object, which is located in the first X-ray projection image, that no item of detection information may therefore be provided if the, for example, detected object in the first X-ray projection image is not confirmed by a corresponding detection in the at least one second X-ray projection image. The detected object is thus discarded. Alternatively, the item of detection information is provided that describes that the at least one object is not identified in a plurality of X-ray projection images. This is an advantageous procedure for preventing incorrect items of detection information.

One exemplary embodiment includes that the function is embodied as a learned function or as an unlearned function. If it is embodied as the unlearned function, it may be embodied, for example, as a rule-based algorithm. The learned function may be taught, that is to say trained, for example, by the first object-ascertaining algorithm and the second object-ascertaining algorithm.

One aspect of the disclosure relates to a computer-implemented method for providing a learned function. The learned function may be suitable in the framework of the above-described computer-implemented method for providing an item of detection information that at least describes whether at least one object is identified in a plurality of X-ray projection images. The computer-implemented method includes receiving training input data that includes at least groups of received and/or artificially generated X-ray projection images that respectively describe a common examination region of an examination object from different directions of projection. The artificially generated X-ray projection images may be understood as synthetic or computer-generated X-ray projection images. One respective group may be a pair of two X-ray projection images or may include more than two X-ray projection images. In addition, in one example, the training input data for the respective group may include an item of relative arrangement information that describes a relative arrangement of the different directions of projection to one another. Alternatively, or in addition, the training input data may include annotated images in which, for example, a predefined category or class from one group of a plurality of categories or classes is assigned to each image pixel. The respective category or class from the group of a plurality of categories or classes may differ, for example, at least between object and non-object. Alternatively, or in addition, on the basis of the category or class, it is possible to identify the object as a bone fragment, internal bleeding, a foreign body, in particular an implant, contrast agent, for example, in a vessel, and/or as a different kind of object in which an individual, such as a physician, is interested and/or which is predefined as the object of interest.

The computer-implemented method includes receiving comparison output data, which may alternatively be referred to as training output data, with the received comparison output data depending on the training input data. The comparison output data for the respective group includes an item of comparison detection information that at least describes whether at least one object is identified in a plurality of X-ray projection images of the group. The item of comparison detection information may be understood as an item of detection information output during teaching or learning of the function. An item of information described by the item of comparison detection information may match, in terms of a type or definition of the described information, an item of information described by the item of detection information. In one example, the comparison output data may include a generated three-dimensional image with at least one object or without an object for the respective group. The object may be the above-described object, such as a bone fragment.

The computer-implemented method includes ascertaining training mapping data by applying a function, which has not been learned yet, to the training input data. Learning or teaching the function, which has not been learned yet, then takes place on the basis of a comparison of the training mapping data with the comparison output data, so the learned function, which is generated by the learning or teaching, is embodied to identify at least one object that is actually present in at least one X-ray projection image. In one example, the computer-implemented method therefore includes learning or teaching a function at least on the basis of the training input data and the comparison output data, so the learned function is embodied to identify at least one object that is actually present in at least one X-ray projection image. The learned function exists as a result of this method act. The computer-implemented method also includes providing the learned function.

The teaching or learning, in particular the training, of the function may be based, in particular, on a comparison of the training mapping data and training output data, which exists here as comparison output data, with the training mapping data having been provided by applying the function to the training input data. The comparison output data may have been provided, for example, by applying an unlearned function, which is different from the function, which has not been learned yet, in particular by applying rule-based algorithms, to the training input data. At least the first object-ascertaining algorithm and the second object-ascertaining algorithm may be taken into account in an alternative example when the function is being taught or learned, for example, in that the first object-ascertaining algorithm and/or the second object-ascertaining algorithm is/are applied and the data generated in this connection, which may be understood as least some of the comparison output data, is compared with the training mapping data. The neural network module includes at least one artificial neural network. The neural network module is a trained machine learning model. Further, the other described algorithms, such as the third object-ascertaining algorithm, the detection image region-ascertaining algorithm, the algorithm for ascertaining the item of probability information, and/or the body part model-ascertaining algorithm, are used to teach or train the function in a training process. The training process with the aid of at least one of the algorithms may include a data comparison between training mapping data, provided by applying the function to training input data, and comparison output data, provided by applying at least one of the algorithms to the training input data. In an example, the received X-ray projection images as well as the item of relative arrangement information are fed into the neural network module. An output of the neural network module may be the item of detection information and/or the information that no item of detection information may be ascertained. It may also be provided that in the case of ascertaining the item of relative arrangement information, an algorithm for ascertaining the item of relative arrangement information when training the learned function, in particular the neural network module, is taken into account. In this example, only the received X-ray projection images are transferred to the learned function, in particular fed into the neural network module.

In an example, the neural network module used includes at least one generative neural network that may be referred to as a Generative Adversarial Network (GAN).

The learned function, for example, the neural network module, was trained in advance to carry out the acts of the computer-implemented method, which it may carry out. At least part of the learned function, which carries out the task of the first object-ascertaining algorithm, may be trained on the basis of synthetic X-ray images virtually rendered from sectional images (for example, computed tomography CT and/or Cone Beam Computed Tomography, CBCT). A ground truth segmentation then exists for each synthetic image pair including sectional image and virtually rendered synthetic X-ray image. This describes, for example, whether and where bone splinters or other clinical findings, such as internal bleeding or contrast agent accumulations may be. Furthermore, real projection images may be annotated in connection with a corresponding volume image, and then in the framework of the training.

Input data for the training may include real and/or synthetically generated projection image pairs, optionally restricted (cropped) to respective epipolar search regions.

The input data may also transfer an epipolar geometry between the X-ray projection images as further input parameters. Input data for the training (training input data) or comparison output data, in particular data for checking the learned function after or during the training, may alternatively or additionally include an annotation of projection images, for example, X-ray projection images, in which image regions are assigned to at least one class or category of object by the annotation.

An architecture of the learned function, for example, a network architecture of the neural network module, may take into account the fact that the generating function or convolutional function respectively accesses only data along the and/or in the vicinity of the corresponding epipolar line in the other X-ray projection image respectively of the plurality of X-ray projection images. The input data may include associated three-dimensional X-ray projection image data or details with and/or without bone fragments or other structures to be detected. If present, at least the bone fragments are at least partially co-annotated in the input data.

The training may include: a generative network as a neural network module is trained, on the basis of the (for example, optionally spatially cut along epipolar lines) X-ray projection image pairs, to generate a three-dimensional image with bone fragment without bone fragment. In the training of the neural network module, a discriminator function is trained to differentiate real objects from generated ones, in particular bone fragments. In addition to the (or as part of the) discriminator function, epipolar consistency with the X-ray projection image pairs from the discriminator function is checked via back projection. A certain haziness may be permitted with the consistency, in particular above a threshold value since exact matches between the back-projected generated three-dimensional images and the projection images are unlikely. Solely the consistency of specific image features may also be checked in the back projection.

Input data during the runtime includes: X-ray projection image pair, possibly respectively restricted to specific epipolar search regions (all relevant image regions are successively given in the neural network module while different embodiments of the generative neural network or else the architecture of the neural network includes the parameterizable consideration of the epipolar search regions), and/or the epipolar geometry between the two X-ray projection images.

The generative network may also be executed multiple times during the runtime for the same epipolar search region, with varied noise being added to the input data. Different synthetically generated three-dimensional image details respectively may consequently be generated in the different embodiments.

Output data during the runtime may include generated three-dimensional image detail, which contains either no bone fragment or at least one generated bone fragment, and/or information on whether or not a bone fragment was generated. The output data may alternatively or additionally take place via a separate detection function that is applied to the generated three-dimensional image detail.

Via a back projection of the generated three-dimensional image details to the projection image pairs, it is possible to check how consistent the generated three-dimensional objects, in particular bone fragments, are with the X-ray projection image pairs. With a consistency above a threshold value, the generated object, in particular bone fragment, is adopted as a candidate in a results list of the neural network module.

In one example, it is not possible for candidate features to be identified in advance in an individual two-dimensional X-ray projection image. Instead, three-dimensional objects, in particular bone fragments, which cannot be detected separately in the individual two-dimensional X-ray projection images, may be generated here by the generative neural network module. The candidates for objects are filtered as described above. Since the method also functions without clearly visible and thus detectable objects in the individual views, (i.e., in individual X-ray projection images), false positives are to be assumed that may be filtered out as described above, for example, by applying the third object-ascertaining algorithm.

A further aspect relates to a method for providing an item of detection information, as was described above. This method includes capturing the first X-ray projection image of the plurality of X-ray projection images and capturing at least one second X-ray projection image of the plurality of X-ray projection images by an X-ray imaging system. The X-ray projection images map an at least partially common examination region of an examination object from different directions of projection. The X-ray projection images are therefore captured from mutually different perspectives. The above-described computer-implemented method is then carried out and, for example, the item of detection information is provided if the condition for this is met. The computer-implemented method is carried out on the basis of the previously captured and subsequently received X-ray projection images. The method is therefore not restricted solely to the processing of the received X-ray projection images but may also include the capturing thereof. The method is carried out, for example, by the X-ray imaging system.

One exemplary embodiment of the method provides that after providing the item of detection information, a marking that describes the at least one detected object is ascertained. The first X-ray projection image and/or the at least one second X-ray projection image is/are displayed, overlaid with the ascertained marking, by a display apparatus. The display apparatus may be part of the X-ray imaging system or be separate from it. For example, one of the X-ray projection images is optically displayed. However, it is displayed in such a way that the detected object is marked in it. The marking may be, for example, a delimitation of the object and/or a box around the object, which is marked in the respective X-ray projection image. The physician or the medical member of staff may therefore view the object, which is potentially of interest, in one or more of the X-ray projection image(s) and, for example, decide whether a further X-ray projection recording may be captured and/or whether other measures may be taken. The described display takes place, in particular, only after the item of detection information has been provided, i.e., if the detected object was detected in a plurality of X-ray projection images. It may be sufficient that, for example, with three X-ray projection images, the object was detected in two of the X-ray projection images, so the item of detection information is then provided and the described display takes place. The object is displayed for further manual choices by the medical member of staff, and this represents a meaningful measure after the item of detection information has been provided.

One further exemplary embodiment of the method includes that, after providing the item of detection information, a notification is ascertained by evaluating the item of detection information, and the ascertained notification is output by an output apparatus, in particular the display apparatus. In particular, the evaluation of the item of detection information may include identifying whether an object was identified in the plurality of X-ray projection images, wherein the notification may be output as a function of an evaluation result. The notification includes that it is recommended that at least one further X-ray projection image is captured from at least one further perspective. Alternatively, or in addition, a three-dimensional X-ray projection recording may be recommended, e.g., a CT scan or a recording with the C-arm X-ray imaging system, in which X-ray projection images are captured at numerous positions along the C-arm. It may therefore be pointed out that detailed X-ray projection images are meaningful before a diagnosis is made and/or a therapy is determined for the patient. In one example, the computer-implemented method may be carried out again on the basis of the further captured X-ray projection images. Consequently, numerous reactions or further uses of the item of detection information are possible in order to assist, particularly conveniently for the physician or the medical member of staff, with the evaluation of the X-ray projection images.

One aspect relates to a data processing system. Unless stated otherwise, all acts of the computer-implemented method may be carried out by the data processing system that includes at least one data processing device. In particular, the at least one data processing device is configured to execute the acts of the computer-implemented method. For this purpose, the at least one data processing device may store, for example, a computer program including commands that, when they are executed by the at least one data processing device, prompt the at least one data processing device to execute the computer-implemented method. The computer-implemented method may also be wholly or partially implemented in terms of hardware. The expressions “data processing system” and “at least one data processing device” may be used interchangeably here and below. This also applies to corresponding expressions derived therefrom.

For the case where the at least one data processing device includes two or more data processing devices, specific acts carried out by the at least one data processing device may also be understood such that different data processing devices carry out different acts or different parts of an act. In particular, it is not necessary for each data processing device to carry out the acts. In other words, the implementation of the acts may be shared among the two or more data processing devices.

A data processing device may refer to a data processing device that includes a processing circuit. The data processing device may therefore process, in particular, data for carrying out computing operations. These may also include operations in order to carry out indicated instances of access to a data structure, for example, a look-up table, LUT, as well as a data processing process implemented in terms of hardware.

The data processing device may include one or more computer(s), one or more microcontroller(s), and/or one or more integrated circuit(s), for example, one or more application-specific integrated circuits, ASIC, one or more field programmable gate arrays, FPGA, and/or one or more system(s) on a chip, SoC. The data processing device may also include one or more processor(s), for example, one or more microprocessor(s), one or more central processing unit(s), CPU, one or more graphics processing unit(s), GPU, and/or one or more signal processor(s), in particular one or more digital signal processor(s), DSP. The data processing device may also include a physical or a virtual group of computers or other of the units.

In different exemplary embodiments, the data processing device includes one or more hardware and/or software interface(s) and/or one or more memory unit(s).

A memory unit may be embodied as a volatile data memory, for example, as a dynamic random access memory, DRAM, or static random access memory, SRAM, or as a non-volatile data memory, for example, as a read-only memory, ROM, as a programmable read-only memory, PROM, as an erasable programmable read-only memory, EPROM, as an electrically erasable programmable read-only memory, EEPROM, as a flash memory or flash EEPROM, as a ferroelectric random access memory, FRAM, as a magnetoresistive random access memory, MRAM, or as a phase-change random access memory, PCRAM.

Receiving or obtaining data or items of information within the meaning of the disclosure may include receiving or obtaining the data, in particular by the data processing system from a transmitting entity, or reading the data from a data memory or receiving or obtaining a data stream that contains the data, or extracting the data from the data stream, etc. In particular, wired or wireless data transmission may be used for this purpose. In particular, the data may be transmitted between a hardware and/or software interface of the transmitting entity and a hardware and/or software interface of the data processing system.

Providing data or items of information may include that the data or items of information is/are available to the data processing system, so it may access the data or items of information. The provided data or items of information may be evaluated or analyzed, for example, by the data processing system and/or by applying an evaluation algorithm. Providing the data may make it possible for the data or items of information to be displayed as a graphical representation and/or may at least contribute to a signal being provided, for example, an actuation signal for a further facility, in particular an X-ray imaging system.

According to a further aspect, a computer program with commands is disclosed. When the commands are executed by a data processing system, the commands prompt the data processing system to carry out a computer-implemented method.

The commands may be in the form, for example, of program code. The program code may be provided, for example, as binary code or Assembler and/or as a source code of a programming language, (e.g., C), and/or as a program script, (e.g., Python).

According to a further aspect, a computer-readable storage medium is disclosed, in particular a physical and/or non-volatile computer-readable storage medium, which stores a computer program.

The computer program and the computer-readable storage medium respectively are computer program products with the commands.

Each exemplary embodiment of the computer-implemented method produces a corresponding exemplary embodiment of the method, which is not purely computer-implemented in that, for example, corresponding acts for generating the X-ray projection images or the acts after providing the item of detection information are incorporated. The disclosure includes combinations of the described exemplary embodiments. The exemplary embodiments of the computer-implemented method are valid, where applicable, for the data processing system and the computer program product.

As described above, the learned function, in particular the neural network module, which may alternatively be understood as a trained machine learning model (MLM), may be provided as the function. A trained MLM may reenact cognitive functions that humans associate with another human mind. In particular, by way of training on the basis of training data, the MLM may be capable of adapting to new circumstances and detecting and extrapolating patterns. Another term for a trained MLM is “trained function.”

In certain examples, the parameters of an MLM may be adjusted or updated by way of training. In particular, supervised training, semi-supervised training, unsupervised training, reinforcement learning and/or active learning may be used. Furthermore, representation learning, which is also referred to as feature learning, may be used. In particular, the parameters of the MLM may be iteratively adjusted by a plurality of acts of the training. In particular, a specific loss function, which is also referred to as a cost function, may be minimized during training. During training of an artificial neural network, ANN, the backpropagation algorithm, in particular, may be used.

An MLM may include an ANN, a support vector machine, a decision tree, and/or a Bayesian network, and/or the MLM may be based on k-means clustering, Q-learning, genetic algorithms, and/or association rules. In particular, an ANN may be or include a deep neural network, a convolutional neural network, CNN, or a convolutional deep neural network. In addition, an ANN may be an adversarial network, a deep adversarial network, and/or a generative adversarial network, GAN.

Further features and combinations of features of the disclosure may be found in the Figures and their description as well as in the claims. In particular, further embodiments of the disclosure do not necessarily have to include all features of one of the claims. Further embodiments may have features or combinations of features that are not mentioned in the claims.

### BRIEF DESCRIPTION OF THE DRAWINGS

FIG. 1 shows a schematic representation of an example of an X-ray imaging system.

FIG. 2 shows a schematic representation of an example of a first X-ray projection image.

FIG. 3 shows a schematic representation of an example of a second X-ray projection image.

FIG. 4 shows a schematic representation of an example of a predefined epipolar region in a first X-ray projection image.

FIG. 5 shows a schematic representation an example of a signal flow graph of a method for providing an item of detection information.

FIG. 6 shows a schematic representation of an example of an epipolar line in a plurality of X-ray projection images.

FIG. 7 shows a schematic representation of an example of an artificial neural network.

### DETAILED DESCRIPTION

The disclosure is explained in more detail below on the basis of specific exemplary embodiments and associated schematic drawings. In the Figures, identical or functionally identical elements may be provided with the same reference numeral. The description of identical or functionally identical elements may not necessarily repeated in respect of different Figures.

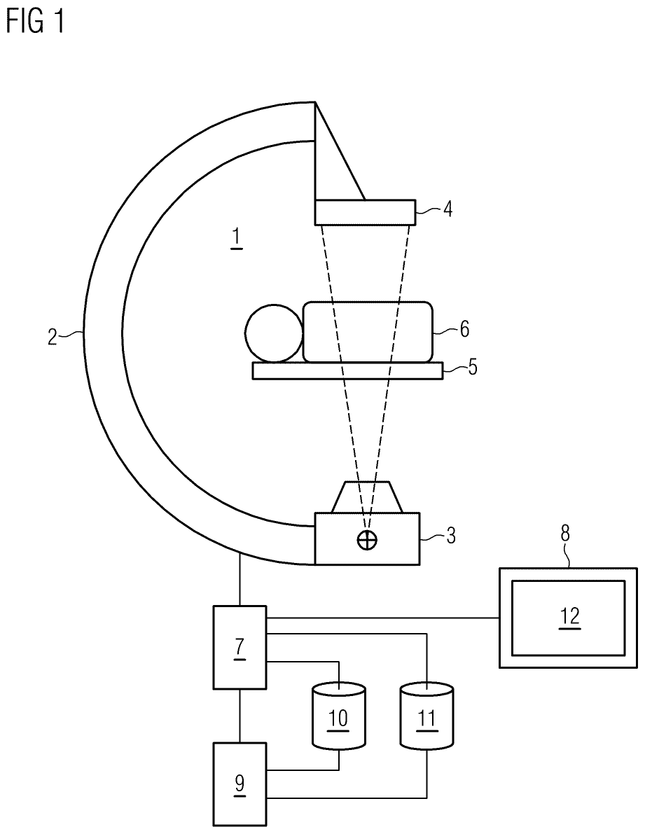

FIG. 1 schematically shows an exemplary embodiment of an X-ray imaging system 1. The X-ray imaging system 1 includes a source unit 3 with an X-ray source, a detector unit 4 with an X-ray detector, and a control system 7 configured to actuate the X-ray source and the X-ray detector in order to generate X-ray images that map an X-ray object 6, for example, a patient.

The X-ray imaging system 1 may include a patient table 5 on which the X-ray object 6 is arranged. The X-ray imaging system 1 includes a data processing system 9 configured to execute a computer-implemented method. Some functions and method acts may be described below, which are executed by the control system 7, while other functions and method acts are described, which are executed by the data processing system 9. The functions and method acts may also be distributed differently in alternative embodiments.

The control system 7 may thus set, for example, different imaging parameters of the X-ray imaging system 1, including, for example, exposure parameters such as a peak kilovoltage of the X-ray source, a tube current of the X-ray source, and/or an X-ray pulse duration. The control system 7 may set further imaging parameters, such as the filter material and/or the filter thickness of an X-ray filter, for example, a copper filter, in that it introduces the corresponding X-ray filter into the beam path or removes it from the beam path. The control system 7 may set further imaging parameters such as the size of the collimator opening of an X-ray collimator. For example, the control system 7 may bring the X-ray collimator into the beam path or remove it from the beam path. The control system 7 may set further imaging parameters, such as an amplification factor of the X-ray detector. The control system 7 may thus bring an anti-scattering grid into the beam path or remove it from the beam path.

In some implementations, the X-ray imaging system 1 includes a display apparatus 8, with the control system 7 being configured to actuate the display apparatus 8 to display an X-ray image 12, a processed X-ray image 12, and/or an image as a function of the processed X-ray image 12. If a medical intervention with fluoroscopy is carried out, the X-ray images are generated, for example, as a sequence of successive images, so an operator may monitor or oversee the medical intervention.

In some implementations, the X-ray imaging system 1 is embodied as a fluoroscopy system, in particular as a C-arm fluoroscopy system, in which the source unit 3 and the detector unit 4 are mounted opposite one another on a C-arm 2 that may be rotated about different axes. The corresponding movements are referred to as an angular or orbital movement. In some embodiments, the patient table 5 and the C-arm 2, aside from the rotational movement of the C-arm 2, may be positioned relative to another by appropriate translation movements of the C-arm 2 and/or the patient table 5. The position and/or the orientation of the X-ray source in relation to the X-ray object 6 and the position and/or the orientation of the X-ray detector in relation to the X-ray object 6 may consequently be set exactly to the desired image perspective.

In some embodiments, the X-ray imaging system 1, for example, the data processing system 9, may include databases 10, 11 for storing and/or reading out data during the course of the computer-implemented method and/or for other purposes.

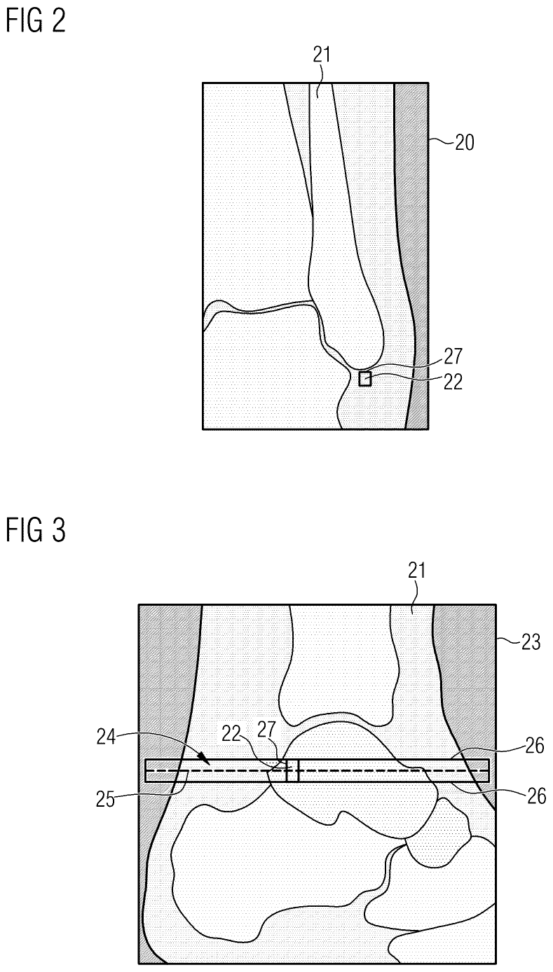

FIG. 2 shows a first X-ray projection image 20. The first X-ray projection image 20 may be the above-described X-ray image 12. The first X-ray projection image 20 describes a body part 21 of the patient, here, by way of example, a bone. The first X-ray projection image 20 is a single two-dimensional X-ray image 12. An object 22 was detected in the first X-ray projection image 20. To highlight this object 22, a marking 27 in the form of a box is drawn around the object 22.

FIG. 3 shows the same body part 21 from a different perspective, represented by a second X-ray projection image 23. The second X-ray projection image 23 is also a single two-dimensional X-ray image 12. In the second X-ray projection image 23, the object 22 from the first X-ray projection image 20 is also detected in the example drawn, and this is highlighted here by the marking 27. It is clear that the object 22 is situated here in a predefined epipolar region 24 that is defined by an epipolar line 25. Two boundary lines 26 delimit the predefined epipolar region 24, for example, parallel to the epipolar line 25.

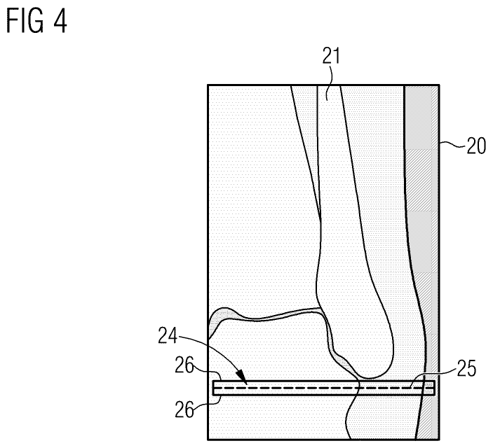

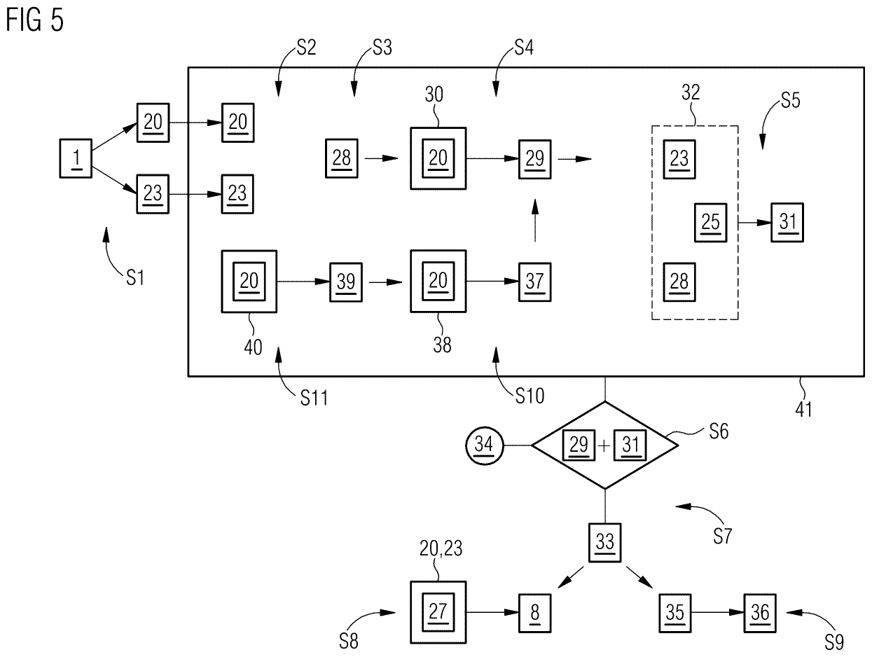

FIG. 4 shows the first X-ray projection image 20, although the predefined epipolar region 24 is drawn around the epipolar line 25 in this image. This corresponds to an epipolar region 24 in which the object 22 drawn in FIG. 2 is arranged (not drawn here).

FIG. 5 shows method acts of a method for providing an item of detection information 33. The item of detection information 33 describes at least whether the at least one object 22 is identified and thus detected in a plurality of X-ray projection images 20, 23. The item of detection information 33 may also describe the object 22. The method may be carried out, for example, on the basis of the X-ray projection images 20, 23, as shown in FIG. 2 to FIG. 4. For example, in a method act S1, the first X-ray projection image 20 and the second X-ray projection image 23 are captured by the X-ray imaging system 1. The X-ray projection images 20, 23 map an at least partially common examination region of an examination object from different directions of projection, which is to say they have been captured from mutually different perspectives. The examination region is, for example, a body part and the examination object is the patient. More than one second X-ray projection image 23 may be captured and taken into account. In the present case, the method is described, purely by way of example, on the basis of exactly one first X-ray projection image 20 and exactly one second X-ray projection image 23.

The first X-ray projection image 20 and the second X-ray projection image 23 are received in a method act S2. The method acts S2, S3, S4, S5, S6, S7, S10 and S11 may be understood as part of a computer-implemented method that may be encompassed by the method in one example.

An item of relative arrangement information 28 is received and/or ascertained in method act S3. This describes a relative arrangement of the different directions of projection to one another.

The method may include applying a function 41 to the received first X-ray projection image 20, which at least one received second X-ray projection image 23 and the received and/or ascertained item of relative arrangement information 28 as input data. The function 41 may be embodied to identify one projection respectively of the at least one object 22 in a predefined epipolar region 24. When the function 41 is applied, one projection respectively of at least one object 22 may therefore be sought and identified in the predefined epipolar region 24, for example, in the first X-ray projection image 20 as well as in the at least one second X-ray projection image 23. The predefined epipolar region 24 is arranged, for example, around an epipolar line 25 on which the projection of the at least one object 22 is situated in the first X-ray projection image 20 and/or in the at least one second X-ray projection image 23. The method may include providing the item of detection information 33 as the output data of the function 41. The provided item of detection information 33 describes that the at least one object 22 is identified in a plurality of X-ray projection images 20, 23 if the projection of the at least one object 22 was identified in the predefined epipolar region 24 in the first X-ray projection image 20 and in the at least one second X-ray projection image 23. If the projection of the at least one object 22 is found in the predefined epipolar region 24 in the first X-ray projection image 20 and in the at least one second X-ray projection image 23, for example, a corresponding item of detection information 33 may therefore be provided.

An exemplary embodiment of the method is described below in detail. In this exemplary embodiment, a first item of object information 29 is ascertained in a method act S4 by applying a first object-ascertaining algorithm 30 to the first X-ray projection image 20. The first item of object information 29 describes the at least one object 22, which is detected in the first X-ray projection image 20.

A second item of object information 31 may be ascertained in a method act S5 in that a second object-ascertaining algorithm 32 different from the first object-ascertaining algorithm 30 is applied to the second X-ray projection image 23, the first item of object information 29 and the item of relative arrangement information 28. The second item of object information 31 describes that the at least one object 22, which was detected in the first X-ray projection image 20, is also detected in the second X-ray projection image 23. Applying the second object-ascertaining algorithm 32 provides, for example, that at least one three-dimensional model of the at least one object 22 is ascertained in the first X-ray projection image 20 on the basis of a projection of this object 22. A projection of the at least one ascertained three-dimensional model is accordingly sought in the predefined epipolar region 24 in the at least one second X-ray projection image 23. If the projection of the at least one ascertained three-dimensional model is found in the second X-ray projection image 23, the object 22 is deemed as also having been detected in the at least one second X-ray projection image 23.

In method act S6, it may be checked whether the first item of object information 29 as well as the second item of object information 31 are ascertained. If this is the case, the item of detection information 33, which describes that the at least one object 22 is identified in a plurality of X-ray projection images 20, 23, is provided, for example, in method act S7. However, if, for example, only the first item of object information 29 or not even the first item of object information 29 is ascertained, discarding 34 may take place, that is to say the method may be terminated, for example, without providing the item of detection information 33, or the item of detection information 33, which describes that the at least one object 22 is not identified in a plurality of X-ray projection images 20, 23, is provided.

After providing the item of detection information 33, a marking 27 describing the detected object 22 may be ascertained, for example, in a method act S8 and this marking 27 may be overlaid on the first X-ray projection image 20 and/or the second X-ray projection image 23 and be displayed by a display apparatus 8. For example, a mapping as in FIG. 2 or FIG. 3 may therefore be displayed.

After providing the item of detection information 33, a notification 35 may be ascertained, for example, by evaluating the item of detection information 33, and be output by an output apparatus 36, in particular by the display apparatus 8, in a method act S9. The notification 35 includes, for example, the recommendation that at least one further X-ray projection image 20, 23 is captured from at least one further perspective and/or that a three-dimensional X-ray projection recording is captured.

After receiving the first X-ray projection image 20 in method act S2, at least one detection image region 37 may be ascertained in method act S10 by applying a detection image region-ascertaining algorithm 38 to the first X-ray projection image 20. The ascertained at least one detection image region 37 specifies at least one image region of the first X-ray projection image 20 for applying the first object-ascertaining algorithm 30. For example, the first object-ascertaining algorithm 30 is then applied in method act S4 to the ascertained detection image region 37, in particular it is only applied to the ascertained detection image region 37.

Beforehand, a three-dimensional body part model 39 may be ascertained by applying a body part model-ascertaining algorithm 40 to at least the first X-ray projection image 20, in particular also to the second X-ray projection image 23, for example, in method act S11. The ascertained three-dimensional body part model 39 three-dimensionally describes a part of the body part 21 mapped in the at least first X-ray projection image 20. The ascertained three-dimensional body part model 39 may be taken into account when the detection image region-ascertaining algorithm 38 is applied in method act S10.

Alternatively, or in addition, the object 22 described by the first item of object information 29 may be registered in the three-dimensional body part model 39. A group of objects of interest may also be known, for which, in addition, one arrangement region respectively is predefined in the body of the patient. An item of probability information may then be ascertained that describes a probability with which the respective projection of the object 22 in the first X-ray projection image 20 and the at least one second X-ray projection image 23 is an object 22 from the predefined group of objects of interest. Only if this ascertained item of probability information is in a predefined probability value range, for example, in method act S7, is the item of detection information 33 provided that describes that the at least one object 22 is identified in a plurality of X-ray projection images 20, 23. Otherwise this may be discarded, for example, as being insufficiently plausible. Alternatively, the function 41 may specify the provided item of detection information 33 as a function of the ascertained item of probability information. Alternatively, or in addition, the provided item of detection information 33 may include the ascertained item of probability information.

When ascertaining this item of probability information, it is also possible to take into account whether an arrangement of the object 22 drawn in the ascertained three-dimensional body part model 39 is at least partially situated in at least one of the arrangement regions that is predefined for at least one of the objects 22 of the predefined group of objects of interest, or not. The three-dimensional body part model 39 may therefore be taken into account as well.

After ascertaining the first item of object information 29, another third object-ascertaining algorithm (not drawn here) may be applied to the first X-ray projection image 20. The epipolar line 25 is then ascertained on which the projection of the at least one object 22 is situated in the first X-ray projection image 20. It is ascertained whether at least one projection of a further object 22, which corresponds with the at least one object 22, is arranged in the predefined epipolar region 24 around the epipolar line 25. If the at least one projection of the further object 22 is ascertained, the item of detection information 33 may not be provided, or that the item of detection information 33 may be provided that describes that the at least one object 22 is not identified in a plurality of X-ray projection images 20, 23. For example, the discarding 34 of the object then occurs in the former case.

Alternatively, if the at least one projection of the further object 22 is ascertained, it is possible to check whether the projection of the at least one object 22 or the projection of the further object 22 may be assigned to the found projection of the at least one ascertained three-dimensional model in the at least one second X-ray projection image 23.

If the projection of the at least one object 22 or the projection of the further object 22 may be assigned to the found projection of the at least one ascertained three-dimensional model in the at least one second X-ray projection image 23, in this example, the item of detection information 33 is provided that describes that the at least one object 22 is identified in a plurality of X-ray projection images 20, 23.

If the projection of the at least one ascertained three-dimensional model is not found in the at least one second X-ray projection image 23, i.e., if no second item of object information 31 may be ascertained in method act S5, in method act S7, no item of detection information 33 is provided or the item of detection information 33 is provided that describes that the at least one object 22 is identified in a plurality of X-ray projection images 20, 23. For example, discarding 34 of the object takes place in the former case.

At least the first object-ascertaining algorithm 30 and the second object-ascertaining algorithm 32 are encompassed by the function 41, in particular by a neural network module, which includes at least one artificial neural network, or the function 41 is taught or trained on the basis of at least the first object-ascertaining algorithm 30 and the second object-ascertaining algorithm 32.

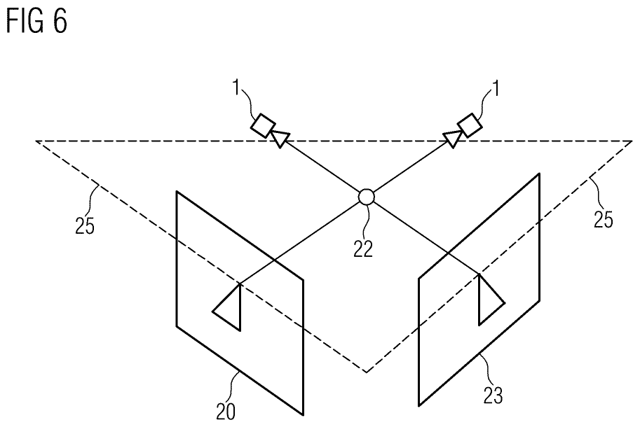

FIG. 6 illustrates the ascertaining of the epipolar region 24 in which the object 22 is sought in the second X-ray projection image 23 and/or further objects 22 are sought in the first X-ray projection image 20. FIG. 6 shows the X-ray imaging system 1 in two different relative positions based on the actual object 22 and thus the patient. It is clear that the object 22 ascertained from different perspectives is arranged in the first X-ray projection image 20 as well as in the second X-ray projection image 23. Owing to the geometry, the projections in the individual X-ray projection images 20, 23 are arranged on a common line that is the epipolar line 25. The epipolar line 25 is consequently a line on which the projection of the object 22 is situated in the first X-ray projection image 20.

During application, for example, of the second object-ascertaining algorithm 32, the predefined epipolar region 24 may be selected in such a way that it is arranged around the ascertained epipolar line 25. In one example, the predefined epipolar region 24 extends up to a boundary line 26 on at least one side of the epipolar line 25. The distance between the epipolar line 25 and the boundary line 26 may be strictly predefined or at least be dependent on a size of the projection of the ascertained three-dimensional model. In an example, boundary lines 26 are situated on both sides of the epipolar line 25, as is drawn, by way of example, in FIG. 3 and FIG. 4.

A computer-implemented method for providing a learned function 41 may be provided. This method may be understood as a learning method or training method for learning or teaching a function 41 as the learned function 41. The computer-implemented method includes receiving training input data that includes at least groups of received and/or artificially generated X-ray projection images 20, 23, which describe a common examination region respectively of an examination object from different directions of projection. In addition, it may include receiving comparison output data that depends on the training input data, with the comparison output data for the respective group including an item of comparison detection information that at least describes whether at least one object 22 is identified in a plurality of X-ray projection images 20, 23 of the group. The computer-implemented method may include ascertaining training mapping data by applying a learned function 41, which has not been learned yet, to the training input data. In addition, it may include learning the learned function 41, which has not been learned yet, on the basis of a comparison of the training mapping data with the comparison output data, so the learned function 41 is embodied to identify at least one object 22 that is actually present in at least one X-ray projection image 20, 23. The computer-implemented method also includes providing the learned function 41.

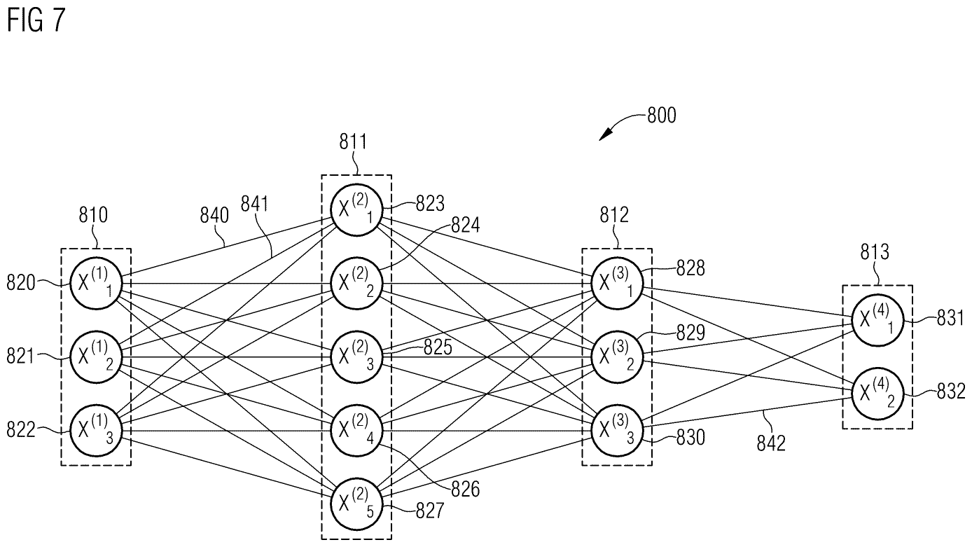

FIG. 7 shows an embodiment of an artificial neural network, ANN, 800, which may be encompassed by the function 41 embodied as a learned function 41. The ANN 800 includes nodes 820, . . . , 832 and edges 840, . . . , 842, wherein each edge 840, . . . , 842 is a directed connection from a first node 820, . . . , 832 to a second 820, . . . , 832. In certain examples, the first node 820, . . . , 832 and the second node 820, . . . , 832 are different nodes 820, . . . , 832. However, it also possible that the first node 820, . . . , 832 and the second node 820, . . . , 832 are identical. In FIG. 7, for example, the edge 840 is a directed connection from the node 820 to the node 823 and the edge 842 is a directed connection from node 830 to node 832. An edge 840, . . . , 842 from a first node 820, . . . , 832 to a second node 820, . . . , 832 is also referred to as an incoming edge for the second node 820, . . . , 832 and as an outgoing edge for the first node 820, . . . , 832.

In this example, the nodes 820, . . . , 832 of the ANN 800 may be arranged in layers 810, . . . , 813, with it being possible for the layers to have an intrinsic order that is introduced by the edges 840, . . . , 842 between the nodes 820, . . . , 832. In particular, the edges 840, . . . , 842 may exist only between adjacent layers of nodes. In the example shown, there is an input layer 810, which includes only the nodes 820, . . . , 822 without incoming edges, an output layer 813, which includes only the nodes 831, 832 without outgoing edges, and hidden layers 811, 812 between the input layer 810 and the output layer 813. In certain examples, the number of hidden layers 811, 812 may be randomly selected. With a multilayer perceptron, MLP, this number is at least one. The number of nodes 820, . . . , 822 inside the input layer 810 relates, as a rule, to the number of input values of the artificial neural network 800, and the number of nodes 831, 832 inside the output layer 813 refers, as a rule, to the number of output values of the artificial neural network 800.

In particular, a real number may be allocated as a value to each node 820, . . . , 832 of the artificial neural network 800. In this case, x⁽ⁿ⁾ᵢ; designates the value of the i^{th} node 820, . . . , 832 of the n^{th} layer 810, . . . , 813. The values of the nodes 820, . . . , 822 of the input layer 810 correspond to the input values of the artificial neural network 800. The values of the nodes 831, 832 of the output layer 813 correspond to the output value of the artificial neural network 800. In addition, each edge 840, . . . , 842 may have a weight that is a real number. In particular, the weight is a real number within the interval [−1, 1] or within the interval [0, 1]. In this case, w^{(m,n)}_{i,j} designates the weight of the edge between the i^{th} nodes 820, . . . , 832 of the m^{th} layer 810, . . . , 813 and the j^{th} node 820, . . . , 832 of the n^{th} layer 810, . . . , 813. In addition, the abbreviation w⁽ⁿ⁾_{i,j} is defined for the weight w^{(n,n+1)}_{i,j}. To calculate the output values of the neural network 800, the input values, in particular, are propagated by the neural network 800. In particular, the values of the nodes 820, . . . , 832 of the (n+1)^{th} layer 810, . . . , 813 may be calculated on the basis of the values of the nodes 820, . . . , 832 of the n^{th} layer 810, . . . , 813 as

$${x}_{j}^{( n + 1 )} = f ( {\sum }_{i} {x}_{i}^{( n )} {w}_{i , j}^{( n )} ) .$$

Here, the function f is referred to as a transfer function or activation function. Known transfer functions are step functions, sigmoid functions, for example, the logistical function, the generalized logistical function, the hyperbolic tangent, the inverse tangent function, the error function, the smooth-step function or rectifier functions. The transfer function is used, for example, for normalization. In particular, the values are propagated layer by layer through the neural network 800, with the values of the input layer 810 being given by the input of the neural network 800, with it being possible to calculate the values of the first hidden layer 811 on the basis of the values of the input layer 810 of the neural network 800, with it being possible to calculate the values of the second hidden layer 812 on the basis of the values of the first hidden layer 811, etc.

To define the values w^{(m,n)}_{i,j} for the edges, the neural network 800 has to be trained with training data. The training data includes, in particular, training input data and training output data (designated as tᵢ). In a training act, the neural network 800 is applied to the training input data in order to generate calculated output data. In particular, the training data and the calculated output data include a number of values that corresponds to the number of nodes of the output layer. In particular, a comparison between the calculated output data and the training data is used in order to recursively adjust the weights within the neural network 800 (backpropagation algorithm). In particular, the weights are changed according to the following formula

$${w}_{i , j}^{′ ( n )} = {w}_{i , j}^{( n )} - γ {δ}_{j}^{( n )} {x}_{j}^{( n )} ,$$

where γ is a predefined learning rate, and the numbers δ⁽ⁿ⁾ⱼ may be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

on the basis of δ⁽ⁿ⁺¹⁾ⱼ if the (n+1)^{th} layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)^{th} layer is the output layer 813, where f′ is the first derivative of the activation function, and t⁽ⁿ⁺¹⁾ⱼ is the comparison training value for the j^{th} node of the output layer 813.

Overall, the examples show an identification of bone fragments via epipolar restrictions. A detection of faintly visible structures (object 22) is carried out on the basis of a plurality of two-dimensional X-ray recordings (X-ray projection images 20, 23) from different perspectives using the epipolar geometry. In particular, anatomical positional relations are taken into account in order to filter out the majority of the false positive findings.

Prerequisites for the method are a first and second X-ray image 12 (X-ray projection images 20, 23), which map an at least partially common part of an anatomy from different projection perspectives. The method may include receiving the at least two X-ray images (X-ray projection images 20, 23).

The method may include receiving or ascertaining the relative projection geometry (item of relative arrangement information 28) between the X-ray images (X-ray projection images 20, 23). The method may include receiving, for example, the angulation data of a C-arm. The method may include ascertaining, for example, via the creation of an artificial intelligence-based, three-dimensional bone model (shape model) on the basis of the X-ray images (X-ray projection images 20, 23) and deriving the relative projection geometry from it. In additional examples, the method may include ascertaining via tracking the patient with a camera and creating, derived therefrom, a three-dimensional model of the patient at the recording instants of the X-ray images (X-ray projection images 20, 23), as well as of the X-ray device.

In certain examples, the method may include creating a three-dimensional model of the bone anatomy on the basis of the X-ray images (X-ray projection images 20, 23), ascertaining anatomical search regions for the detection task (detection image region 37). The anatomical search regions may be a vicinity of the soft tissue around bone or around the regions of a joint or in the vicinity of a fracture that is directly visible in the two-dimensional X-ray projection images 20, 23.

In certain examples, the method may include image-based detection of possible fragments in the first X-ray projection image 20 (candidate, here object 22).

In certain examples, the method may include a search in the second X-ray projection image 23 in the epipolar region 24 around the epipolar line 25 of the candidate (object 22) for corresponding features to this candidate (object 22), which may be understood as correspondence candidates.

In certain examples, the method may include a search along the epipolar lines 25 in the first X-ray projection image 20 for whether, in addition to the original candidate (object 22), there are further possible corresponding features to the correspondence candidates.

In certain examples, the method may include discarding 34 candidates (objects 22) for which no correspondence candidates were found, or in which there were other corresponding features. Optional filtering-out of plausible candidates (objects 22) using the three-dimensional model. Bone fragments or other findings are only to be expected in specific regions of the anatomy, for example, in the region of joints. In particular, in the three-dimensional space, they are not to be expected inside bone, but rather outside, but in the vicinity, of the bone.

In certain examples, the method may include displaying the candidates (objects 22) as an overlay or marking 27 in the two-dimensional X-ray projection images 20, 23. The physician accordingly has, for example, three choices: discarding 34 the candidates (objects 22); recording at least one additional two-dimensional X-ray projection image 20, 23, with this having a new perspective of the anatomy or an increased radiation dose in order to improve the image quality, as well as repeating the method using the additional X-ray projection image 20, 23; three-dimensional X-ray recording, for example, computed tomography (CT) or Cone Beam Computed Tomography (CBCT).

The challenge compared with known methods, which include the search for corresponding features, is that the shape of the unknown bone fragment, which is to say the object 22, must firstly be estimated. This means that a modelling is made based on the object 22 found in the first X-ray projection image 20 and based on what size and shape the associated object, in particular bone fragment, may have. This results in the ascertainment of the three-dimensional model.

For example, a diameter and/or a direction of the projection may be detected in the first X-ray projection image 20 and a shape and/or size of the object 22, for example, of the bone fragment, may be derived from this. Furthermore, depending on image contrast of the detected projection, a thickness of the object 22 (bone fragment) along the direction of projection may also be estimated. Back projections of this estimated three-dimensional shape of the object 22 (bone fragment) are ascertained with the projection geometry of the second X-ray projection image 23, with the position along the epipolar line 25 in the second X-ray projection image 23 being taken into account (the positioning and size of the estimated three-dimensional object 22 in the space, and the direction of penetration slightly, changes as a result). In the second X-ray projection image 23, a projection, which is compatible with the back projection (for example, in shape/size/image contrast), is respectively sought along the epipolar line 25.

As a further possibility, the function 41 for the detection of the candidates for bone fragments (objects 22) may also be trained to indicate a size (and possibly further shape parameters) of the bone fragment (object 22) in addition to the location of the detected candidate (object 22). These parameters may then be scaled, altered, and/or adjusted accordingly as a function of the position along the epipolar line 25 in the second X-ray projection image 23.

To summarize, the disclosure relates in one example to a computer-implemented method and a method for providing an item of detection information 33 that describes an object 22 detected in a plurality of X-ray projection images 20, 23, including: receiving a first X-ray projection image 20 and at least one second X-ray projection image 23, which were captured from mutually different perspectives; ascertaining a first item of object information 29 that describes an object 22 that is detected in the first X-ray projection image 20; ascertaining a second item of object information 31 that describes that the object 22 is also detected in the second X-ray projection image 23, wherein at least one three-dimensional model of the object 22 is ascertained on the basis of a projection of the object 22 in the first X-ray projection image 20 and a projection of the at least one ascertained three-dimensional model is sought in a predefined epipolar region 24 in the second X-ray projection image 23, wherein at least if the projection is found, the object 22 is also detected in the second X-ray projection image 23; and if the first item of object information 29 and the second item of object information 31 are ascertained, providing the item of detection information 33.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing an item of detection information, the method comprising:
receiving a first X-ray projection image of a plurality of X-ray projection images and at least one second X-ray projection image of the plurality of X-ray projection images, wherein the first X-ray projection image and the at least one second X-ray projection image map an at least partially common examination region of an examination object from different directions of projection;
receiving and/or ascertaining an item of relative arrangement information that describes a relative arrangement of the different directions of projection to one another;
applying a function to the first X-ray projection image, the at least one second X-ray projection image, and the item of relative arrangement information as input data, wherein the function is embodied to identify one projection respectively of at least one object in a predefined epipolar region in the first X-ray projection image and in the at least one second X-ray projection image, and wherein the predefined epipolar region is arranged around an epipolar line on which the projection of the at least one object is situated in the first X-ray projection image and/or in the at least one second X-ray projection image; and
providing the item of detection information as output data of the function, wherein the item of detection information describes that the at least one object is identified in the plurality of X-ray projection images when the projection of the at least one object is identified in the predefined epipolar region in the first X-ray projection image and in the at least one second X-ray projection image.

2. The method of claim 1, wherein the function comprises a first object-ascertaining algorithm and a second object-ascertaining algorithm, and
wherein the applying of the function comprises:
ascertaining a first item of object information by applying the first object-ascertaining algorithm to the first X-ray projection image, wherein the first item of object information describes at least one object detected in the first X-ray projection image;
ascertaining a second item of object information by applying the second object-ascertaining algorithm to the at least one second X-ray projection image, the first item of object information, and the item of relative arrangement information, wherein the second item of object information describes that the at least one object, which was detected in the first X-ray projection image, is also detected in the at least one second X-ray projection image, wherein the second object-ascertaining algorithm is configured to ascertain at least one three-dimensional model of the at least one object based on a projection of the at least one object in the first X-ray projection image and to identify a projection of the at least one three-dimensional model in the at least one second X-ray projection image in a predefined epipolar region, and wherein when the projection of the at least one three-dimensional model is identified in the at least one second X-ray projection image, the at least one object is also detected in the at least one second X-ray projection image; and
providing the item of detection information that describes that the at least one object is identified in the plurality of X-ray projection images only when both the first item of object information and the second item of object information are ascertained.

3. The method of claim 2, further comprising:
ascertaining the epipolar line on which the projection of the at least one object is situated in the first X-ray projection image, and
wherein, when the second object-ascertaining algorithm is applied, the predefined epipolar region in the at least one second X-ray projection image is arranged around the epipolar line.

4. The method of claim 3, wherein the predefined epipolar region extends at least on one side of the epipolar line up to a boundary line, and
wherein a distance between the epipolar line and the boundary line is predefined or is at least dependent on a size of the projection of the at least one three-dimensional model.

5. The method of claim 2, further comprising:
ascertaining, after the first X-ray projection image has been received, at least one detection image region by applying a detection image region-ascertaining algorithm to the first X-ray projection image,
wherein the at least one detection image region predefines at least one image region of the first X-ray projection image for applying the first object-ascertaining algorithm.

6. The method of claim 5, further comprising:
ascertaining, after the first X-ray projection image has been received, a three-dimensional body part model by applying a body part model-ascertaining algorithm to at least the first X-ray projection image,
wherein the three-dimensional body part model three-dimensionally describes a body part described by the first X-ray projection image, and
wherein the three-dimensional body part model is taken into account when the detection image region-ascertaining algorithm is applied.

7. The method of claim 2, further comprising:
ascertaining an item of probability information after ascertaining the second item of object information, wherein the item of probability information describes a probability with which the respective projection of the object in the first X-ray projection image and in the at least one second X-ray projection image is an object of interest from a predefined group of objects, and
wherein the function only provides the item of detection information, which describes that the at least one object is identified in the plurality of X-ray projection images, when the item of probability information is situated in a predefined probability value range or the function specifies the item of detection information as a function of the item of probability information and/or the item of detection information comprises the item of probability information.

8. The method of claim 7, further comprising:
ascertaining a three-dimensional body part model by applying a body part model-ascertaining algorithm to at least the first X-ray projection image,
wherein the three-dimensional body part model three-dimensionally describes a part of a body of the examination object mapped by the first X-ray projection image, and the object is registered with the three-dimensional body part model,
wherein, for objects in the predefined group of objects of interest, at least one arrangement region respectively is predefined in the body, and
wherein, when ascertaining the item of probability information, it is taken into account whether an arrangement of an object registered in the three-dimensional body part model is situated at least partially in the at least one arrangement region, which is predefined for at least one of the objects in the predefined group of objects of interest.

9. The method of claim 2, further comprising:
applying, after ascertaining the first item of object information, a third object-ascertaining algorithm to the first X-ray projection image;
ascertaining an epipolar line on which the projection of the at least one object is situated in the first X-ray projection image; and
ascertaining, when at least one projection of a further object that corresponds with the at least one object is ascertained, whether the at least one projection of the further object is arranged in a predefined epipolar region around the epipolar line,
wherein no item of detection information is provided or the provided item of detection information describes that the at least one object is not identified in the plurality of X-ray projection images, or
wherein it is checked whether the projection of the at least one object or the projection of the further object is assignable to the projection of the at least one three-dimensional model in the at least one second X-ray projection image, and wherein, when the projection of the at least one object or the projection of the further object is assignable, the item of detection information is provided that describes that the at least one object is identified in the plurality of X-ray projection images.

10. The method of claim 2, wherein, when the projection of the at least one three-dimensional model is not found in the at least one second X-ray projection image, no second item of object information is ascertained and the item of detection information is provided that describes that the at least one object is not identified in the plurality of X-ray projection images.

11. The method of claim 1, wherein the function is embodied as a learned function.

12. The method of claim 1, further comprising:
capturing the first X-ray projection image of the plurality of X-ray projection images and the at least one second X-ray projection image of the plurality of X-ray projection images by an X-ray imaging system.

13. The method of claim 12, further comprising:
ascertaining a marking describing the at least one object after providing the item of detection information; and
displaying, by a display, the first X-ray projection image and/or the at least one second X-ray projection image overlaid with the marking.

14. The method of claim 13, further comprising:
ascertaining a notification after providing the item of detection information, wherein the ascertaining comprises evaluating the item of detection information, and wherein the notification comprises a recommendation that at least one further X-ray projection image is captured from at least one further perspective and/or a three-dimensional X-ray projection recording; and
outputting, by the display, the notification.

15. A computer-implemented method for providing a learned function, the method comprising:
receiving training input data comprising at least groups of received X-ray projection images and/or artificially generated X-ray projection images that describe a common examination region respectively of an examination object from different directions of projection;
receiving comparison output data that depends on the training input data, wherein the comparison output data for a respective group comprises an item of comparison detection information that at least describes whether at least one object is identified in a plurality of X-ray projection images of the respective group;
ascertaining training mapping data by applying a function, which has not been learned yet, to the training input data;
learning the function based on a comparison of the training mapping data with the comparison output data, so the learned function is configured to identify at least one object actually present in at least one X-ray projection image; and
providing the learned function.

16. A data processing system comprising:
one or more processors configured to:
receiving a first X-ray projection image of a plurality of X-ray projection images and at least one second X-ray projection image of the plurality of X-ray projection images, wherein the first X-ray projection image and the at least one second X-ray projection image map an at least partially common examination region of an examination object from different directions of projection;
receiving and/or ascertaining an item of relative arrangement information that describes a relative arrangement of the different directions of projection to one another;
applying a function to the first X-ray projection image, the at least one second X-ray projection image, and the item of relative arrangement information as input data, wherein the function is embodied to identify one projection respectively of at least one object in a predefined epipolar region in the first X-ray projection image and in the at least one second X-ray projection image, and wherein the predefined epipolar region is arranged around an epipolar line on which the projection of the at least one object is situated in the first X-ray projection image and/or in the at least one second X-ray projection image; and
providing the item of detection information as output data of the function,
wherein the provided item of detection information describes that the at least one object is identified in the plurality of X-ray projection images when the projection of the at least one object is identified in the predefined epipolar region in the first X-ray projection image and in the at least one second X-ray projection image.
